(19) United States
(12) Patent Application Publication
(10) Pub. No.: US 2023/0050728 A1
(43) Pub. Date: Feb. 16, 2023

(54) METHOD OF MANUFACTURING SILICA MICROSPHERES

(71) Applicant: GRAG TECHNOLOGIES PTY LIMITED, Willoughby (AU)

(72) Inventors: Andrew RUYS, Willoughby (AU); Gregory MCKENZIE, Willoughby (AU); Ronald SLOCOMBE, Willoughby (AU)

(21) Appl. No.: 17/758,560

(22) PCT Filed: Oct. 5, 2021
(86) PCT No.: PCT/AU2021/051158

§ 371 Date: Jul. 8, 2022

(30) Foreign Application Priority Data
Oct. 16, 2020 (AU) 2020903765

(51) Int. Cl. A61K 51/12; A61K 9/16; C01B 33/193

(52) U.S. Cl. CPC A61K 51/1251; A61K 9/1682; A61K 9/1605; C01B 33/193

(57) ABSTRACT

There is provided a method of manufacturing silica microspheres includes the steps of mixing acid and water to form a mixture; adding a silicon alkoxide to the mixture so as to precipitate microspheres; allowing the microspheres to settle into a sediment and removing a supernatant liquid; and immersing the microspheres in acid.

### TECHNICAL FIELD

The present invention relates to silica microspheres and methods for their manufacture. Embodiments of the present invention find application, though not exclusively, for use in medical procedures, such as selective internal radiation therapy (SIRT), for example.

### BACKGROUND ART

Any discussion of documents, acts, materials, devices, articles or the like which has been included in this specification is solely for the purpose of providing a context for the present invention. It is not to be taken as an admission that any or all of these matters form part of the prior art base or were common general knowledge in the field relevant to the present invention as it existed in Australia or elsewhere before the priority date of this application.

One type of commercially available microsphere is polymer-based and is sold by Sirtex under the trademark SIR-Spheres®. These prior art microspheres must be manufactured by infusing porous polymer microparticles (ion exchange microspheres) with a solution of radioactive yttrium. That is, they must be manufactured in close proximity to equipment capable of producing radioactive yttrium-90, such as a neutron-beam (nuclear reactor), or a strontium generator (Sr90/Y90), or other radioactive yttrium-90 producing apparatus. There is also the requirement for the associated complex and expensive infrastructure for handling radioactive solutions and live radioactive products. SIR-Spheres® are essentially yttrium-infused ion exchange microspheres.

When one uses ion exchange resin microspheres as the carrier for the yttrium, being ion exchange microspheres, there is always the risk that under certain conditions of body chemistry, particularly undesirable pH changes, some radioactive yttrium can potentially be leached from the ion exchange microspheres, and travel in the blood to other locations of the body, thereby exposing the patient to the risk of systemic irradiation. Hence, this prior art yttrium-infused ion exchange microsphere is comparatively expensive and difficult to manufacture, and not without its potential clinical risks. An additional complication associated with the use of this type of polymer-based microsphere is that once a dose has undergone radioactive decay significantly beyond its half life, it is radioactively cold thereafter and therefore cannot be used, nor can it be reused. Therefore, if it reaches the patient too late, it can never be used or reused. Advantageously, this type of prior art microsphere has an apparent density when immersed of slightly higher than 1.0 g·cm⁻³. This density is well suited for use in SIRT.

Another type of commercially available microsphere is glass-based and is sold by Boston Scientific under the trademark Therasphere®. These prior art microspheres can be mass-produced in large quantities in a conventional manufacturing facility and then stored for a period of time in a non-radioactive condition. Following storage, they may be despatched in bulk, or in dose portions, to a nuclear reactor for irradiation, and then shipped to hospitals for SIRT therapy. If these ceramic prior art microspheres are not used in time, they can be re-sterilised and re-irradiated for use at a subsequent date. Therasphere® comprises microspheres of yttrium aluminosilicate (YAS) glass.

Glass-bonding is a chemically durable means of containing chemical elements. Therefore, the yttrium is strongly chemically bonded within a YAS microsphere with no risk of leaching. With yttrium-infused ion exchange microspheres, leaching by reversible ion exchange remains a possibility. YAS microspheres need to comply with glass-forming oxide ratios and therefore YAS microspheres inherently contain a very high yttrium load, more than an order of magnitude higher than yttrium-infused ion exchange microbeads. With a much higher yttrium load comes the potential for much higher levels of individual microsphere radioactivity for YAS microspheres, compared to yttrium-infused ion exchange microspheres. This means that for a given clinical radiation dosage by SIRT therapy, less YAS microspheres might be needed, compared to (lower radioactivity) yttrium-infused ion exchange microspheres. Highly radioactive microspheres can potentially expose the blood cells to much higher local radiation dosages, which has the potential to reduce the blood platelet count.

YAS has an apparent density of approximately 3 g·cm⁻³. It has been appreciated by the inventors that this density is higher than is desirable for use in SIRT. Such high densities can cause YAS microspheres to sediment too rapidly out of the blood plasma, before they have reached the tumour. Such high densities can also inhibit the even distribution of the YAS microspheres in the target organ and may cause accumulation in excessive concentrations in parts of the organ that are not cancerous. This can decrease the amount of effective radiation that reaches the cancer in the target organ. Heavy microspheres, particularly microspheres with a density greater than about 2.3 g·cm⁻³, can be difficult to deliver through infusion tubing as they settle within the tubing unless the injection force is substantial and the flow rate of the suspending liquid is high. High pressures and fast delivery flow rates are contra-indicated when infusing radioactive microspheres into the hepatic artery of patients as the microspheres will potentially reflux back into blood vessels such as the gastro-duodenal artery, splenic artery, and left gastric artery. This can result in undesirable consequences. In contrast, lighter microspheres, such as yttrium-infused ion exchange microspheres, can distribute well within the liver.

Hence, it has been appreciated by the inventors of the present application that the manufacture and/or use of each of the above-discussed commercially available prior art SIRT microsphere technologies, YAS and yttrium-infused ion exchange microspheres, entails significant inherent advantages and disadvantages.

### SUMMARY OF THE INVENTION

It is an object of the present invention to overcome, or substantially ameliorate, one or more of the disadvantages of the prior art, or to provide a useful alternative. Essentially this invention combines the benefits of low sphere density and lower yttrium load (principal benefits of SIR-Spheres®) with the benefits of glass bonding of the yttrium (a principal benefit of Therasphere®)

In a first aspect of the present invention there is provided a method of manufacturing silica microspheres, the method including the steps of: mixing acid and water to form a mixture; adding a silicon alkoxide to the mixture so as to precipitate microspheres; allowing the microspheres to settle into a sediment and removing a supernatant liquid; and immersing the microspheres in acid.

In one embodiment the method includes monitoring the temperature of the mixture whilst the microspheres are precipitating and waiting until the temperature is at or near a peak before taking the steps of: allowing the mixture to settle; removing the supernatant liquid; and immersing the microspheres in acid.

In another embodiment the method includes allowing the microspheres to precipitate for a period of between 5 and 25 minutes and then taking the steps of: allowing the mixture to settle; removing the supernatant liquid and immersing the microspheres in acid.

Preferably the method further includes, after immersing the microspheres in acid, allowing the microspheres to settle into a sediment and removing a supernatant liquid. Preferably these immersing, settling and removing steps are each repeated at least once.

Preferably the method further includes immersing the microspheres in water, allowing the microspheres to settle into a sediment and removing a supernatant liquid. Preferably these immersing, settling and removing steps are each repeated at least once.

Preferably the method further includes immersing the microspheres in an alkali, allowing the microspheres to settle into a sediment and removing a supernatant liquid. Preferably these immersing, settling and removing steps are each repeated at least once. In one embodiment the alkali is ammonia. In another embodiment the alkali is sodium hydroxide.

An embodiment of the method further includes drying the microspheres at temperatures of less than 200° C. Preferably this drying includes: immersing the microspheres in water in a container and placing the container in a water bath having a temperature of approximately 90° C. to 100° C. for at least 30 minutes; removing a majority of the supernatant water so as to leave an approximately 1 mm to 5 mm layer of water above the microspheres; placing the microspheres within the container in a dryer at a temperature of between 100° C. and 110° C. for at least 10 hours; progressively raising the temperature of the dryer to a temperature of between 140° C. and 160° C. over a period of approximately 1 hour; and maintaining the microspheres within the container in the dryer at a temperature of between 140° C. and 160° C. for at least 7 hours.

Another embodiment of the method further includes drying the microspheres at temperatures of between 200° C. and 400° C. Preferably this drying includes: drying the microspheres in air at an ambient temperature for between 12 and 36 hours; and placing microspheres in a dryer that is progressively heated to a temperature of between 250° C. and 350° C. over a period of 20 minutes to 40 minutes; and maintaining the microspheres in the dryer at a temperature of between 250° C. and 350° C. for 30 to 90 minutes.

Preferably the method further includes infusing a radionuclide into the microspheres. In one embodiment the radionuclide is a material containing yttrium. More preferably, the step of infusing a radionuclide into the microspheres includes: placing the microspheres into a container; mixing the microspheres with a yttrium-89 nitrate solution; placing the container into a water bath having a temperature of approximately 90° C. to 100° C. for at least 30 minutes; leaving the container in the water bath for at least 10 hours whilst allowing the water bath to cool; removing the supernatant yttrium-89 nitrate solution; adding water, allowing the microspheres to settle and removing supernatant water; and calcining the microspheres.

In some embodiments the step of infusing a radionuclide into the microspheres is repeated at least once. Preferably, prior to a repeating of the step of infusing a radionuclide into the microspheres, the microspheres are calcined at a temperature of between 300° C. and 500° C.

Preferably the step of calcining the microspheres includes: allowing the microspheres to cool; placing the microspheres into a dryer having a temperature of approximately 100° C. to 110° C. for at least 10 hours; placing the microspheres into a furnace and heating the furnace at a rate of approximately 150° C. to 250° C. per hour to a target temperature of approximately 600° C. to 950° C.; and maintaining the microspheres at the target temperature for 30 to 90 minutes.

Preferably the method further includes exposing yttrium-89 infused microspheres to neutron radiation so as to form yttrium-90 infused microspheres. In some embodiments of the method, prior to exposing the yttrium-89 infused microspheres to neutron radiation, the yttrium-89 infused microspheres are stored whilst in a non-radioactive state.

Preferably the silicon alkoxide is tetra ethyl ortho silicate (TEOS).

Preferably the acid is acetic acid.

In a second aspect of the present invention there are provided silica microspheres manufactured in accordance with the method as described above.

In a third aspect of the present invention there are provided silica microspheres for use in a medical procedure, the microspheres being manufactured in accordance with the method described above and being infused with a radionuclide.

Preferably the radionuclide contains yttrium. More preferably, the microspheres have a yttrium load by weight of between approximately 0.1% and 5%. Also preferably, the microspheres are neutron transparent.

In a fourth aspect of the present invention there are provided silica microspheres for use in a medical procedure, the microspheres being manufactured in accordance with the method described above and being infused with a medicament.

In a fifth aspect of the present invention there are provided silica microspheres manufactured in accordance with the method described above wherein the microspheres have an apparent-density-when immersed in the range of approximately 1.2 g·cm⁻³ to 2.2 g·cm⁻³.

According to another aspect of the invention there are provided silica microspheres manufactured in accordance with the method described above wherein the microspheres have a total open porosity in the range of approximately 5% to 40%.

The features and advantages of the present invention will become further apparent from the following detailed description of preferred embodiments, provided by way of example only, together with the accompanying drawings.

### BRIEF DESCRIPTION OF THE ACCOMPANYING DRAWINGS

FIG. 1 is a first flow chart depicting precipitation, arresting and sieving steps in a preferred embodiment of the method;

FIG. 2 is a second flow chart depicting gentle drying steps in the preferred embodiment of the method;

FIG. 2A is an alternative second flow chart depicting harsh drying steps in another preferred embodiment of the method;

FIG. 3 is a third flow chart depicting infusion and calcining steps in the preferred embodiment of the method;

FIG. 4 is a bar graph showing the yield of microspheres in the 20 to 60 micron diameter range;

FIGS. 5 and 6 are bar graphs showing the effects on apparent density (i.e. immersed density after soaking in water at 25° C. for 20 minutes) arising from taking steps that diverge from the standard parameters (the standard parameters, shown as ‘Optimal Process’ on FIG. 5 are a process involving: 5 Molar ammonia double wash; gentle drying; and 21 minute reaction arrest);

FIG. 7 is a logarithmic line of best fit showing the effects of the concentration of the washing ammonia on apparent density;

FIG. 8 is a bar graph showing the effects on apparent density of various calcination methodologies;

FIG. 9 is a graph with a solid line showing actual measured Yttrium Load versus Density for various embodiments of the invention after a single infusion and a dotted line showing the calculated maximum Yttrium Load versus Density that may theoretically be achieved under ideal conditions; and

FIG. 10 is a graph with a thin line showing actual measured Yttrium Load versus Density for various embodiments of the invention after a single infusion and a thick line showing the calculated maximum Yttrium Load versus Density that may theoretically be achieved with multiple infusions, under ideal conditions. By way of comparison, this graph also shows Yttrium Loads versus Densities for prior art Theraspheres and SirSpheres.

### DETAILED DESCRIPTION OF PREFERRED EMBODIMENTS OF THE INVENTION

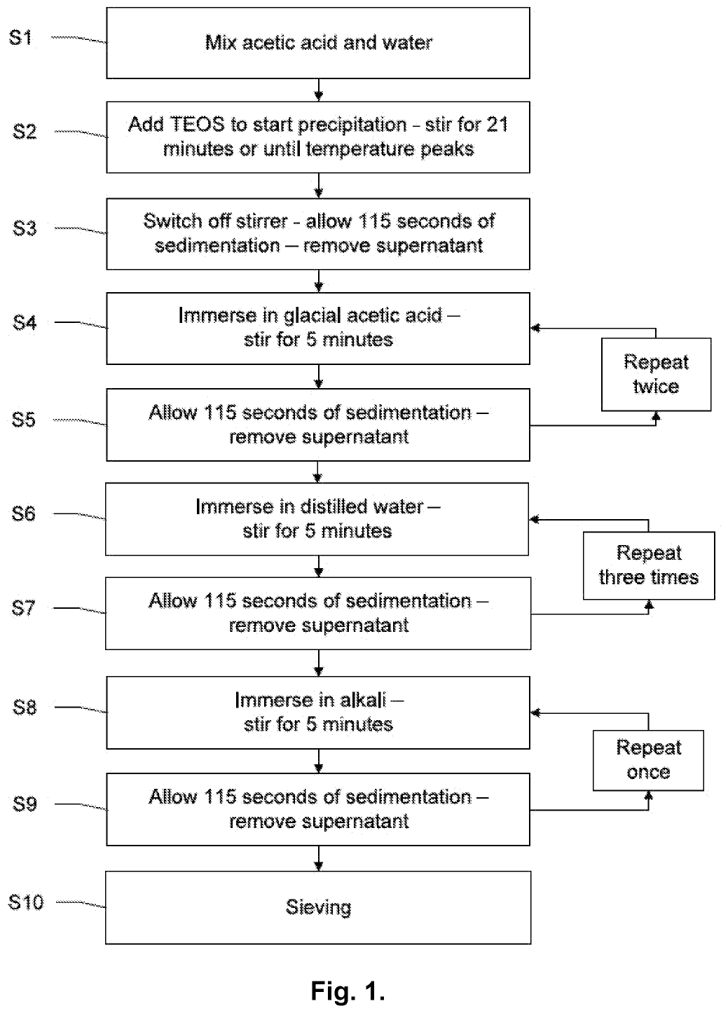

Referring to FIG. 1, an embodiment of the method of manufacturing silica microspheres commences at step S1 with the mixing of 360 g (4 Moles) of acid and 135 g (5 Moles) of water in a 1 litre beaker to form a mixture. It will be appreciated that many acids can be used; however, in the preferred embodiment the acid is acetic acid, which advantageously provides a very low pH with no inorganic residue and is generally suited for microspheres that are proposed to be used in medical procedures. This mixing, along with the other mixing/stirring steps mentioned below, are preferably performed by automated means, such as a magnetic stirrer or an impellor mixer (although in some circumstances an impellor mixer may be preferable if it is found that a magnetic stirrer grinds the particles). The amounts of the various reactants are measured in a 0.001 g precision balance.

Next an amount of 312 g (1 Mole) of a silicon alkoxide, which in the preferred embodiment is tetra ethyl ortho silicate (hereinafter referred to as TEOS) is prepared. In alternative embodiments other silicon alkoxides may be used, for example TMOS (tetramethyl orthosilicate). When expressed in moles, the ratios of the three reactants is 4:5:1. At step S2 the TEOS is added in one sudden motion into the mixture in the beaker. The resulting mixture is then mixed and stirred for between 5 and 25 minutes, with the amount of time in the preferred embodiment being precisely 21 minutes. During this 21 minute period, microspheres of varying diameters precipitate out of the mixture. In other words, the 21 minute precipitation reaction period commences when the TEOS is added into the mixture.

The amount of time for which the microspheres are allowed to precipitate from the mixture is selected to strike a balance between yield and the need to avoid gelling, which occurs if the precipitation is allowed to continue excessively. The 21 minute time period mentioned above has been found to work well with the amounts of reactants mentioned above and when the precipitation occurs in a 1 litre beaker at an ambient temperature of approximately 25° C. However, changes to any of these parameters are likely to cause a change in the optimum precipitation time. A method to determine a suitable precipitation time for a given set of reaction parameters is to use an immersion thermometer to monitor the temperature of the mixture whilst the microspheres are precipitating. The precipitation is allowed to continue until the temperature is at or near a peak. The moment at which the temperature is seen to level out and start to decrease defines the end of the optimum precipitation time. This method of determining precipitation time, which is based upon the exothermic nature of a stage of the precipitation reaction, has been found to be effective at avoiding gelling and to improve the yield of microspheres in the desired size range.

Once the precipitation time has elapsed, at step S3 the stirrer is switched off and the microspheres are given 115 seconds to form a sediment. This 115 second sedimentation time is specific to the arrangement being used and other sedimentation times may be required in other circumstances. More particularly, it will be appreciated by those skilled in the art that the amount of time required for the microspheres to form a sediment will vary depending mainly upon the height of the liquid column through which the particles are descending (i.e. the height of the supernatant) and upon the diameter of the particles. The resulting supernatant liquid is then removed with the use of a suction hose.

At step S4 900 ml of glacial acetic acid is poured into the beaker such that the microspheres are immersed in the glacial acetic acid. It is believed by the inventors that this step commences an arresting of the precipitation reaction. It has been appreciated by the inventors that arresting the reaction at an appropriate point eliminates or reduces gelling risk and browning problems. It also enhances yield in the desired size range, along with the nanoporosity of the microspheres. The magnetic stirrer is switched on and the suspension is stirred for 5 minutes and then the stirrer is turned off.

At step S5 the mixture is allowed 115 seconds of sedimentation time, after which the supernatant liquid is removed by a suction hose. Steps S4 and S5 are then repeated at least once and in the preferred embodiment two more times. Hence, in the preferred embodiment these acid washing steps are performed a total of three times.

At step S6 900 ml of distilled water is poured over the microspheres so as to immerse them in the distilled water. The magnetic stirrer is switched on and the suspension is stirred for 5 minutes and then the stirrer is turned off.

At step S7 the mixture is allowed 115 seconds of sedimentation time, after which the supernatant liquid is removed by a suction hose. Steps S6 and S7 are then repeated at least once and in the preferred embodiment three more times. Hence, in the preferred embodiment these water washing steps are performed a total of four times. These water washing steps are believed to remove the acid from the pores in the micro spheres.

At step S8 an alkali, such as ammonia or sodium hydroxide, is poured over the microspheres so as to immerse them in the alkali. For microspheres that are proposed to be used in a medical procedure, it is generally preferable for ammonia to be used as the alkali It has been appreciated by the inventors that after washing, the ammonia fairly quickly volatilises away, whereas the NaOH remains. Thus, if the NaOH is not removed by multiple washing of the suspension after the alkali curing stage, it remains indefinitely and can possibly have an etching effect. In the preferred embodiment the alkali is 900 ml of 5 Molar ammonia solution having a pH of approximately 12. The magnetic stirrer is switched on and the suspension is stirred for 5 minutes and then the stirrer is turned off.

At step S9 the mixture is allowed 115 seconds of sedimentation time, after which the supernatant liquid is removed by a suction hose. Steps S8 and S9 are then repeated at least once. In the preferred embodiment these alkali washing steps are performed a total of two times. These alkali washing steps are believed to cure the pores in the microspheres. Without these alkali washing steps, the microspheres form into excessively open porous structures. It is believed by the inventors that the ammonia washing increases the prevalence of closed pores by promoting a resistance of the nanoporous structure to “opening up” during drying. This helps to minimise the apparent-density-when-immersed of the microspheres. It is also believed that the mechanism is that the ammonia causes substantially all unreacted silicate precursor in the surface pore openings to react and hydrolyse, sealing off most of the porosity and thereby rendering the majority of the pores in the microspheres closed porosity.

Additionally, these alkali washing steps increase pH, which controls the zeta potential (i.e. attractiveness between microsphere particles) to promote separation between the microspheres, which is believed to help to avoid gelling.

It has been appreciated by the inventors that if one were to proceed straight to the alkali washing steps (S8 and S9) without firstly conducting the acetic acid washing steps (S4 and S5) and the water washing steps (S6 and S7), then the entire microsphere/acetic-acid/water suspension would form a rigid gel causing the microsphere manufacturing process to fail.

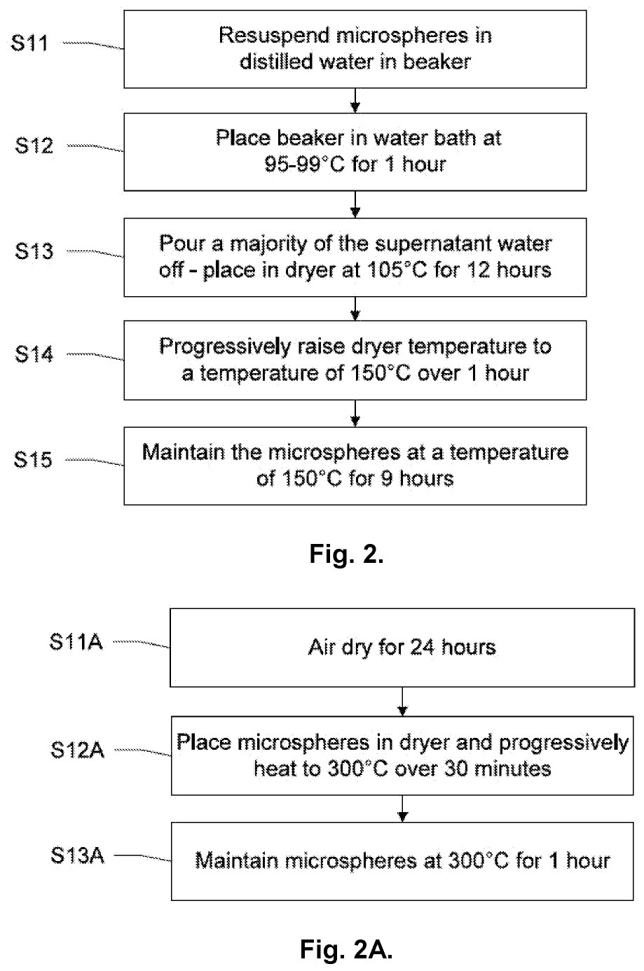

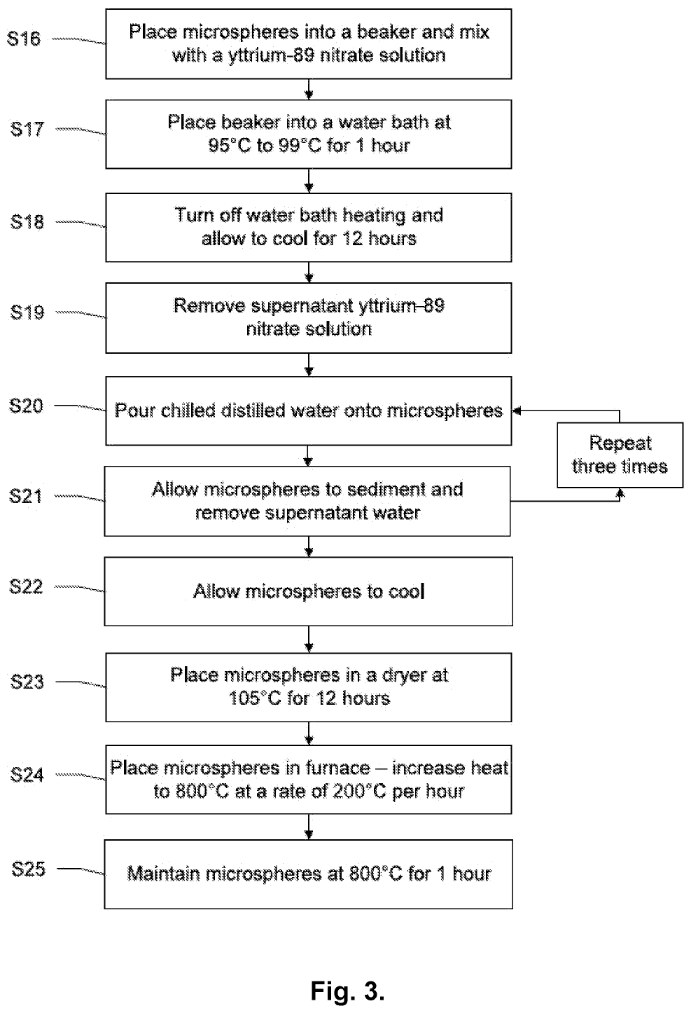

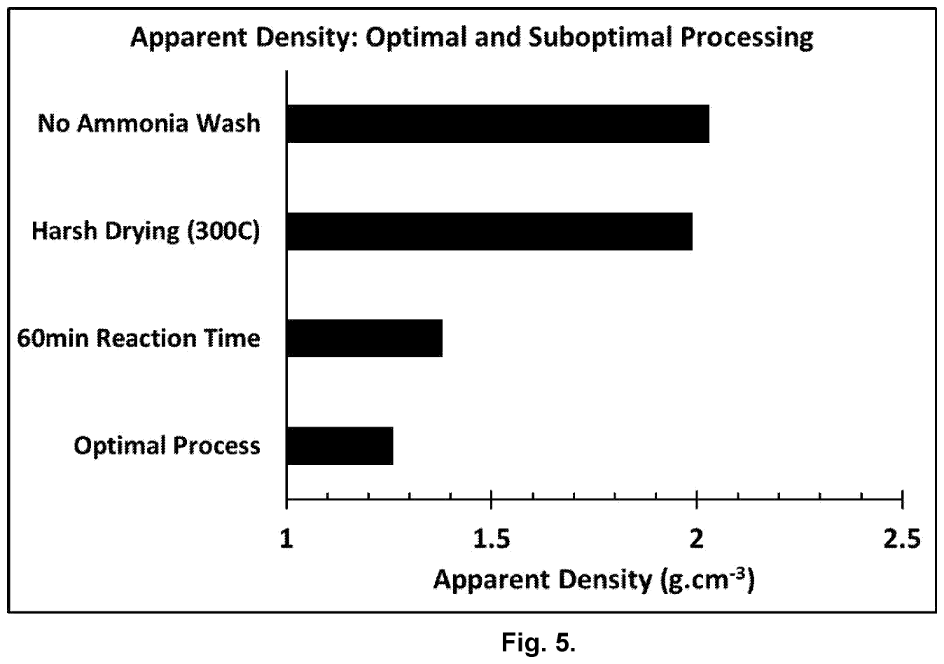

At step S10 a sieving process is used to separate the desired microsphere particle sizes from the unwanted sizes. Although it is possible to introduce a delay at this point, ideally the sieving commences immediately once the precipitation is complete. Depending upon the usage that is intended for the microspheres, the sieving may be tailored to yield only those microspheres having diameters lying in various ranges. For example, the sieving may be tailored to yield only those microspheres having a diameter lying in the range of 5 to 200 microns. Another possible range is 15 to 100 microns. If proposed to be used in a medical procedure such as SIRT, a range of 20 to 60 microns is desirable. Due to the range of sizes that typically form during the precipitation reaction, and depending upon the breadth of the desired range of sizes, it is not unusual for up to 95% of the microspheres to be discarded at this point. As an example, to yield a range of, say, 20 to 60 microns, the microspheres are passed through a 60 micron sieve and the microsphere that are caught in the 60 micron sieve are discarded because that are bigger than 60 microns. Next the microspheres that passed through the 60 micron sieve are passed through a 20 micron sieve and the microsphere that pass through the 20 micron sieve are discarded because that are smaller than 20 microns. The microspheres that are caught in the 20 micron sieve are retained. The yields in the 20 to 60 micron range as determined for various experimental batches of microspheres are depicted in FIG. 4. The process flow now moves onto the dying steps depicted in FIG. 2 or 2A.

FIG. 2 depicts drying steps that may be referred to generally as ‘gentle drying’, which entails drying the microspheres at temperatures of less than 200° C. These gentle drying steps have been found to result in microspheres having minimal apparent-density-when-immersed. This is believed to be because the lower drying temperatures result in lower steam pressures within the pores of the microspheres during drying, which causes minimal disruption to the porous structure. Hence, the microspheres resulting from gentle drying are slower to become waterlogged when immersed, which gives rise to the minimal apparent-density-when-immersed property; however, they are less porous and therefore have a lower capacity for radionuclide infusion. FIG. 2A depicts drying steps that may be referred to generally as ‘harsh drying’, which entails drying the microspheres at temperatures of between 200° C. and 400° C. The higher drying temperatures result in higher steam pressures within the pores of the microspheres during drying, which is believed to cause increased disruption of the porous structure. Hence, the microspheres resulting from harsh drying are faster to become waterlogged when immersed, which gives rise to higher apparent-density-when-immersed properties; however, they are more porous and therefore have a higher capacity for radionuclide infusion. Depending upon the desired characteristics for the microspheres that are to be manufactured, it is possible to devise a drying process that lies somewhere in between the gentle and the harsh approaches shown in FIGS. 2 and 2A respectively.

The gentle drying process shown in FIG. 2 commences at step S11 with resuspending the microspheres in distilled water in a container, such as a 100 ml beaker. At step S12 the container is placed in a water bath having a temperature of approximately 90° C. to 100° C., and more preferably 95-99° C., for at least 30 minutes, and more preferably for 1 hour, to fill the open pores with water.

At step S13 the majority of the supernatant water is poured off to leave an approximately 1 mm to 5 mm, and preferably 2 mm, layer of water above the microspheres. The microspheres contained within the beaker are then placed within a dryer at a temperature of between 100° C. and 110° C., and preferably 105° C. for at least 10 hours, and preferably for 12 hours.

At step S14 the temperature of the dryer is progressively raised to a temperature of between 140° C. and 160° C., and preferably 150° C., over a period of approximately 1 hour. At step S15 the microspheres are maintained within the beaker in the dryer at a temperature of between 140° C. and 160° C., and preferably 150° C., for at least 7 hours, and preferably for 9 hours.

It will be appreciated by those skilled in the art that other gentle drying techniques may be employed in alternative embodiments of the method, for example supercritical drying.

The gentle-dried microspheres have very low 1 and 20 minute apparent-density-when-immersed values of between approximately 1.5 g·cm⁻³ and 1.25 g·cm⁻³. These values are highlighted in the table below. Notably there is little or no difference between the apparent-density-when-immersed values after a 1 minute immersion compared to the values after 20 minutes of immersion. In other words, the microspheres that were subject to gentle drying exhibited an unusually high resistance to water absorption for sustained periods of immersion. To the best knowledge of the inventors, these are the lowest apparent-density-when-immersed values ever recorded for glass/ceramic microspheres.

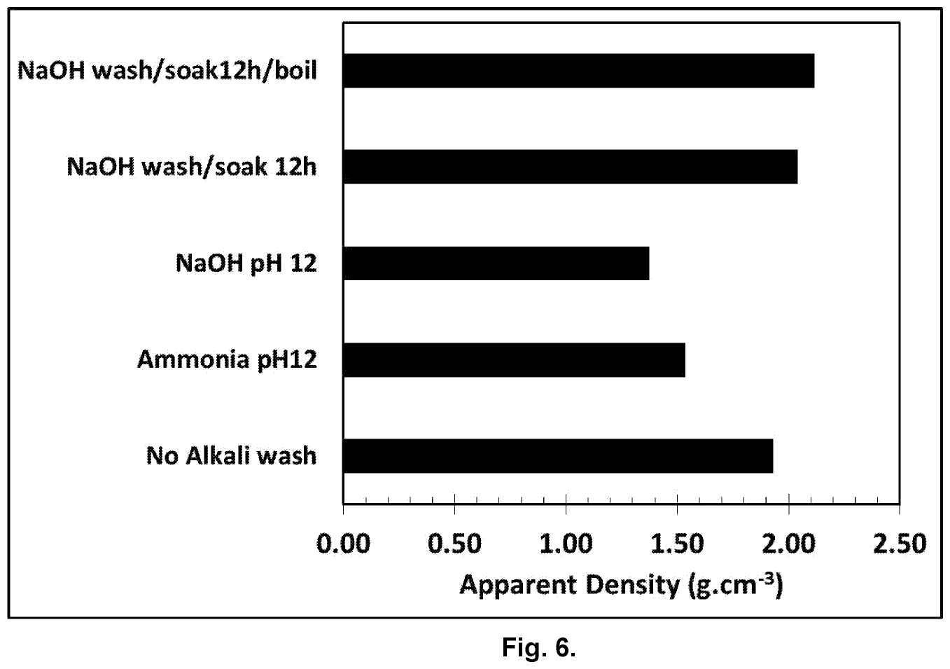

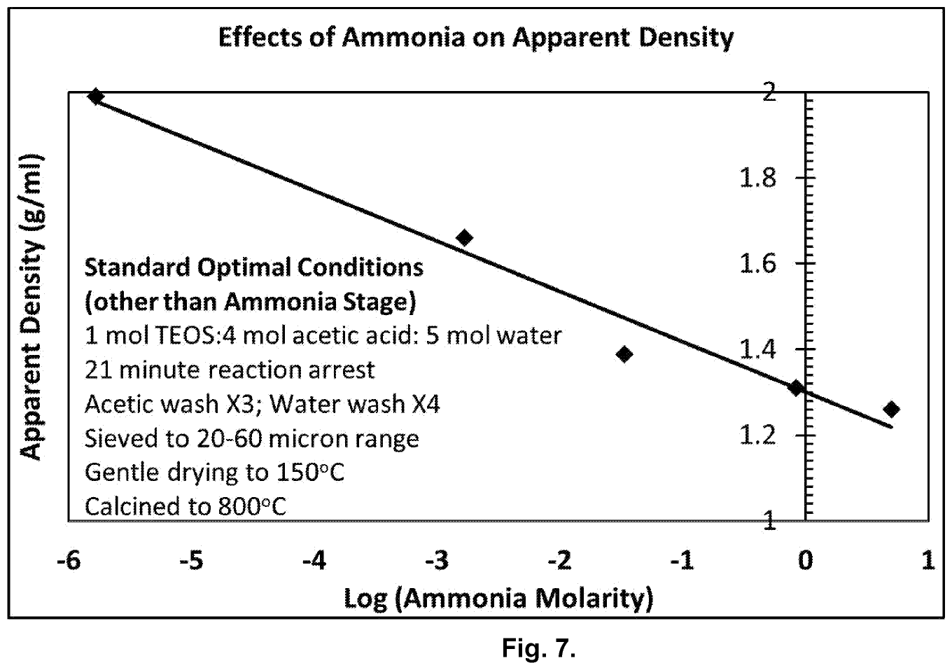

FIGS. 5 and 6 depict the apparent-densities-when-immersed of various experimental batches of microspheres that were manufactured in accordance with the preferred method, with certain departures as listed on the vertical axis. These various methodologies resulted in apparent-densities-when-immersed ranging between approximately 1.25 g·cm⁻³ for the optimal process (i.e. optimal if low density is your main criteria) and approximately 2.2 g·cm⁻³. The effects of the ammonia molarity on the apparent-densities-when-immersed of various experimental batches of microspheres are shown in FIG. 7. Advantageously, an apparent-density-when-immersed of 1.25 g·cm⁻³ is very close to the density of water or blood plasma. To the best of the inventors' knowledge, this is the closest to the density of water, of any ceramic (SIRT) microparticle, in clinical use, by a significant margin.

Various values as measured for three experimental batches of microspheres (referred to as 2216A, 2219A and 2220B) that were prepared using the gentle drying technique are set out in the table below.

| Batch Number | 2216A | 2219A | 2220B |
|---|---|---|---|
| Apparent Density | 1.39 | 1.33 | 1.24 |
| Total Porosity % | 35.9 | 38.6 | 42.9 |
| Immersed Density 1 minute | 1.4 | 1.33 | 1.24 |
| % Porosity Filled: 1 minute | 0.5 | 0 | 0 |
| Immersed Density 20 minute | 1.44 | 1.33 | 1.25 |
| % Porosity Filled: 20 minute | 2.3 | 0 | 0.3 |
| Immersed Density: 60 minute boil | 1.55 | 1.52 | 1.35 |
| % Porosity Filled: 60 minute boil | 7.4 | 8.7 | 5.1 |
| Total Open Porosity % | 7.4 | 8.7 | 5.1 |
| Total Closed Porosity % | 28.6 | 29.9 | 37.8 |

The harsh drying process shown in FIG. 2A commences at step S11A by allowing the microspheres to dry in flowing air at an ambient temperature for between 12 and 36 hours, and preferably for 24 hours. At step S12A the microspheres are placed in a dryer that is progressively heated to a temperature of between 250° C. and 350° C., preferably 300° C., over a period of 20 minutes to 40 minutes, preferably 30 minutes. At step S13A the microspheres are maintained in the dryer at a temperature of between 250° C. and 350° C., preferably 300° C., for 30 to 90 minutes, preferably 1 hour.

High resolution scanning electron microscopy has revealed that the surface pores in both the gentle-dried and the harsh-dried microspheres are approximately 10 nm to 50 nm in diameter. However, the number of surface pores is greatly increased in the harsh-dried microspheres in comparison to the gentle-dried microspheres.

Various values as measured for an experimental batch of microspheres (referred to as 2216B) that was prepared using the harsh drying technique are set out in the table below.

| Batch Number | 2216B |
|---|---|
| Apparent Density | 1.24 |
| % Total Porosity | 42.8 |
| Immersed Density 1 minute 25° C. | 1.94 |
| % Porosity Filled: 1 minute 25° C. | 32.3 |
| Immersed Density 20 minute 25° C. | 2.01 |
| % Porosity Filled: 20 minute 25° C. | 35.5 |
| Immersed Density: 60 minute boil | 2.08 |
| % Porosity Filled: 60 minute boil | 38.7 |
| Total Open Porosity % | 38.7 |
| Total Closed Porosity % | 4.1 |

It can be seen from the above table that the harsh drying technique produced microspheres with a total open porosity of 38.7% when boiled for 1 hour. This was at the expense of 1 and 20 minute apparent-density-when-immersed (compared to that of the gentle dried batches), which was 1.94 g·cm−3 for 1 minute and 2.01 g·cm−3 for 20 minutes. In general, the silica microspheres can be manufactured in accordance with some of the harsh drying embodiments of the preferred method to have a total open porosity in the range of approximately 5% to 40%. To the best of the inventors' knowledge, these harsh-dried microspheres are the only SIRT product capable of combination radionuclide/drug therapy, for example with chemotherapy drugs.

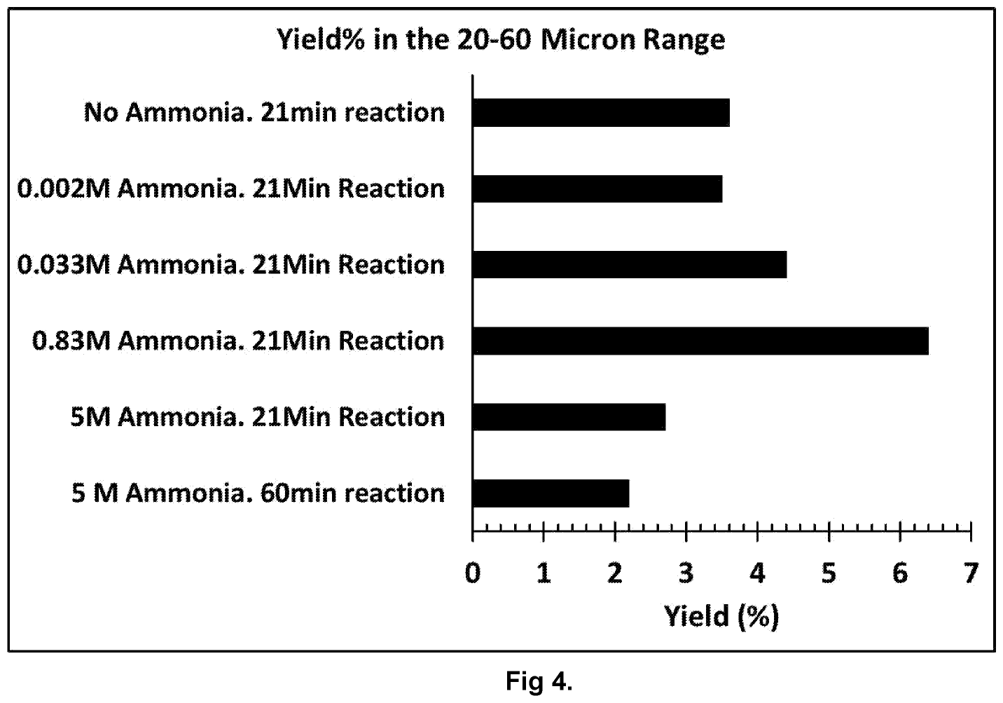

The process flow now proceeds to FIG. 3, which depicts the infusion of a radionuclide, which in the preferred embodiment is a material containing yttrium, into the pores of the microspheres. This commences at step S16 with the placement of an amount, which in one implementation was 15.8 grams, of the microspheres into a 100 ml beaker and then pouring 60 ml of 1.09 molar yttrium-89 nitrate solution onto the microspheres. The yttrium-89 nitrate solution may be prepared by mixing 25.6 g Y(NO3)3.6H2O yttrium nitrate hexahydrate salt, with 50 ml of demineralised water. Examples of other potentially suitable radionuclides include, but are not restricted to, holmium, iodine, phosphorous, iridium, rhenium, samarium or alkoxide solutions thereof (which may be suited for use in anhydrous situations). These alternative radionuclides may be infused into the microspheres using an essentially identical procedure to that used for yttrium. In some situations, it may be desirable to incorporate a second radionuclide, such as one having a specific gamma emission that can be used for procedures such as dosimetry or imaging using a gamma camera. Typically, such a gamma emission will be in addition to the emission of the primary therapeutic radionuclide.

At step S17 the beaker containing the microspheres mixed with the yttrium-89 nitrate solution is placed into a water bath having a temperature of approximately 90° C. to 100° C., preferably 95° C. to 99° C., for at least 30 minutes, preferably 1 hour. This fills the open pores of the microspheres with the yttrium-89 nitrate solution.

At step S18 the heating of the water bath is turned off and the microspheres and yttrium-89 nitrate solution are allowed to remain in the beaker in the in the water bath for at least 10 hours, preferably 12 hours, whilst the water bath cools. At step S19 the supernatant yttrium-89 nitrate solution is removed by pouring it off.

At step S20 120 ml of distilled water (ideally chilled to at or less than 5° C.) is poured onto the microspheres. At step S21 the microspheres are allowed to settle and then the supernatant water is removed. Steps S20 and S21 are then repeated three times (i.e. for a total of four water washes). Alternatively, steps S20 and S21 may be repeated and each time the supernatant water is removed, its electrical resistance may be tested so as to measure its yttrium nitrate concentration. The water washes should continue until the supernatant has a low molarity (e.g. 0.01 or 0.001). These water washing steps (i.e. S20 and S21) are to ensure that substantially no yttrium nitrate remains on the external surface of the microspheres. That is, substantially all yttrium nitrate remaining should be inside the pores of the microspheres.

It is believed by the inventors that infusing the radionuclide into the pores as a concentrated salt solution, under either boiling or autoclave conditions, helps to maximise pore filling. In general, the silica microspheres manufactured in accordance with the preferred method can be engineered to allow for a yttrium load by weight of between approximately 0.1% and 5%. More specifically, the Y2O3 concentration of an experimental batch of the single infusion gentle-dried microspheres was measured by X-ray fluorescence by the University of NSW Mark Wainwright Analytical Centre and found to be 0.17 weight %. This is comparable, but slightly lower, than the highest theoretical yttrium load of a Sir-Sphere®, which is calculated by the inventors to be 0.79 volume % on an equivalent Y2O3 basis (this equates to 0.23 weight % yttrium as Y2O3 for a sphere density of 1.3 g·cm⁻³). In the Sir-Spheres product the yttrium is present as yttrium triphosphate; whereas in the ceramic embodiment, yttrium is present as the oxide Y2O3. The preferred embodiments of the silica gel microspheres as manufactured by the present method, with their 0.17 weight % yttrium oxide load, had a density of 1.29 g·cm⁻³.

The Y2O3 concentration of an experimental batch of the single-infusion harsh-dried microspheres was measured by X-ray fluorescence by the University of NSW Mark Wainwright Analytical Centre and found to be 4.54 weight %. By the inventors' calculations, this equates to approximately 20 times greater than the highest theoretical yttrium load in a Sir-Spheres® microsphere (0.23 weight %).

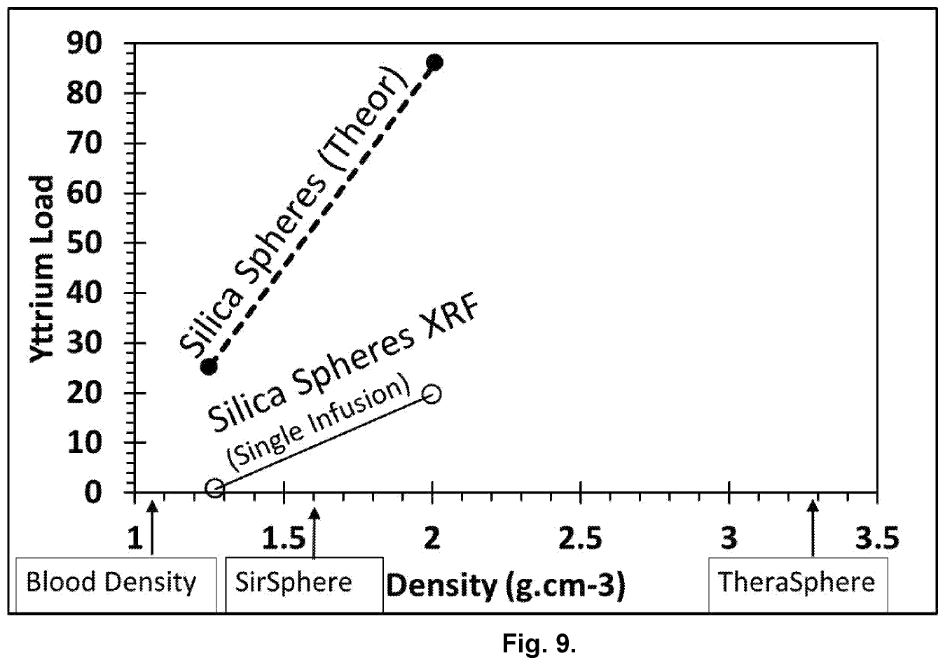

The vertical axes of graphs 9 and 10 depict a normalised yttrium oxide loading in which the yttrium oxide loading of the prior art SirSphere has a value of 1. Two embodiments are shown: a gentle-dried embodiment (density 1.27 g·cm−3) and a harsh-dried embodiment (density 2.0 g·cm−3). The actual measured yttrium oxide loading achieved by a single infusion of a test batch of these microspheres versus the immersed density is shown in the solid line of FIG. 9 (with the yttrium oxide loading values having been determined by X-ray fluorescence spectroscopy). Hence, after a single infusion, microspheres having a density equal to that of the SirSpheres could be expected to have a yttrium oxide loading that is approximately 9 times greater than that of the SirSpheres. The theoretical yttrium oxide loading, under ideal conditions, is shown by the dotted line in FIG. 9. Hence, after a single infusion, microspheres having a density equal to that of the SirSpheres could potentially have a yttrium oxide loading that is approximately 50 times greater than that of the SirSpheres, if infused under ideal conditions, i.e., all available pores fully infused with yttrium salt solution.

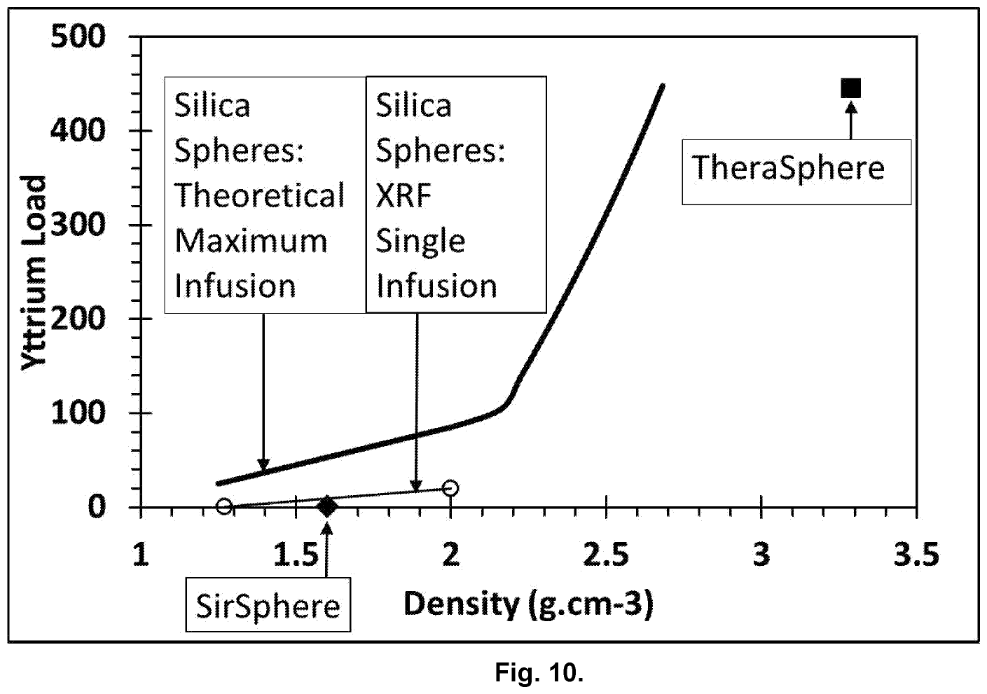

If the yttrium oxide loading yielded by a single infusion is considered insufficient, it is possible in some embodiments of the method to repeat the step of infusing the radionuclide into the microspheres at least once. Each repeating of the infusing steps typically drives the resultant yttrium oxide loading of the microspheres closer to the theoretical maximum loading (as calculated by the inventors), which is shown by the solid line in FIG. 10. Each time the step of infusing the radionuclide into the microspheres is to be repeated, the microspheres are calcined at a temperature of between 200° C. and 800° C., preferably approximately 500° C.

FIG. 10 depicts a similar graph to that shown in FIG. 9, with the actual measured single infusion values from FIG. 9 shown in FIG. 10 by a thin line, and the calculated theoretical maximum values, for multiple infusions, shown in FIG. 10 by the thicker line. Additionally, the yttrium oxide loadings and densities of the prior art SirSpheres and ThersSpheres are depicted on FIG. 10. It can be seen that the particles manufactured by the preferred embodiment of the present invention have the potential to perform substantially better than both the prior art SirSpheres and TheraSpheres with regards to yttrium oxide loading versus immersed density.

The preceding paragraphs describe the infusion of a radionuclide into the nanopores of the microspheres. However, it will be appreciated that instead of a radionuclide, in some circumstances it may be desirable to infuse a medicament, such as ibuprofen or chemotherapy drugs for example, into the nanopores of the microspheres. In yet other circumstances it may be desirable to infuse both a radionuclide and a medicament into the nanopores of the microspheres for use in combination therapy.

Step S22, in which the microspheres are allowed to cool, commences calcination of the microspheres (or, for embodiments in which calcination was performed prior to a repeating of the infusion step, step S22 represents the commencement of the final calcination of the microspheres). Calcination is believed to vitrify the radionuclide in the nanopores of the microspheres, which is important to help ensure that the radionuclide does not leach out in vivo. At step S23 the cool damp microspheres are placed in a dryer having a temperature of approximately 100° C. to 110° C., and preferably 105° C. for at least 10 hours, and preferably for 12 hours. At step S24 the microspheres are placed into a furnace, which is heated at a rate of approximately 150° C. to 250° C. per hour, and preferably 200° C. per hour, to a target temperature of approximately 600° C. to 950° C., preferably 800° C. At step S25 the microspheres are maintained at the target temperature for 30 to 90 minutes, and preferably for 1 hour. This completes the calcining process and, once cool, the microspheres can now be placed into storage (advantageously, in a non-radioactive state) or they can be immediately irradiated for use in a medical procedure (as described in more detail below).

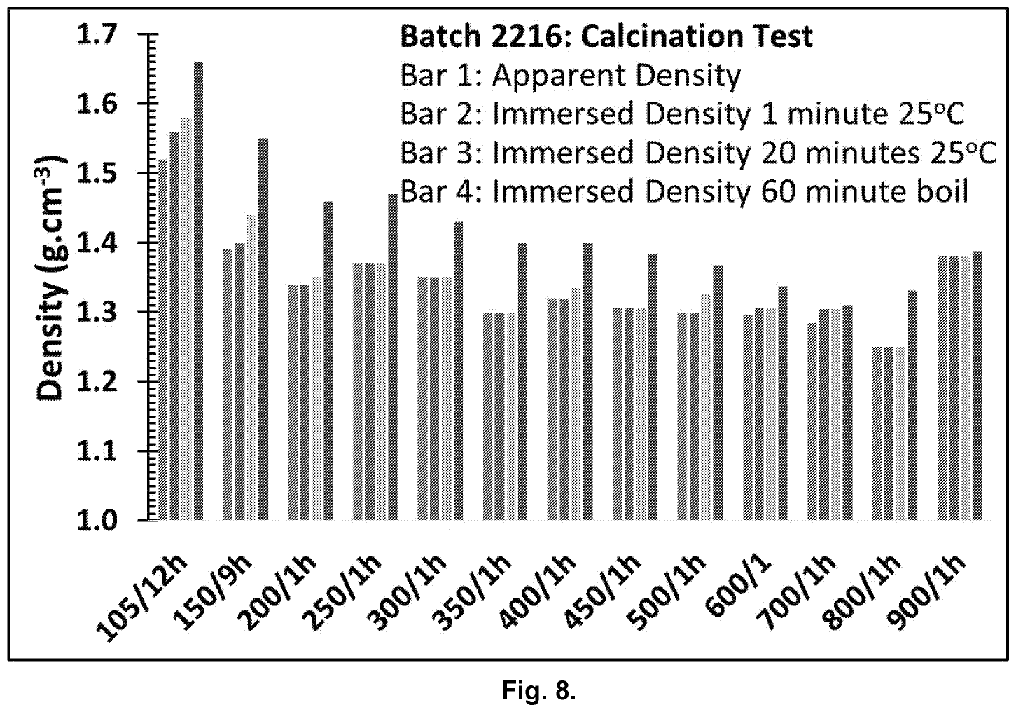

To determine the target temperature of 800° C. mentioned in the preceding paragraph, the inventors ran experiments in which the microspheres were calcined at various temperatures. The process described in the preceding paragraph and depicted in steps S22 to S25 was followed, except differing test target temperatures were utilised as follows: 200° C.; 250° C.; 300° C.; 350° C.; 400° C.; 450° C.; 500° C.; 600° C.; 700° C.; 800° C. and 900° C. Following calcining the microspheres were cooled and tested to determine: apparent density; apparent-density-when-immersed in 25° C. water for 1 minute; apparent-density-when-immersed in 25° C. water for 20 minutes; and apparent-density-when-immersed in boiling water for 60 minutes. The results are graphed in FIG. 8, from which it can be seen that a calcining target temperature of 800° C. resulted in microspheres having the lowest apparent densities. It is also believed by the inventors that a calcining target temperature of 800° C. helps to optimise the bond strength retaining the radionuclide within the nanopores of the micro spheres. Additionally, 800° C. is generally below the temperature at which cristobalite starts to form.

Once it is desired to utilise the microspheres in a medical procedure, such as SIRT for example, the yttrium-89 infused microspheres are irradiated by exposing them to neutron radiation so as to form yttrium-90 infused microspheres. Advantageously, the silica microspheres manufactured in accordance with the preferred method are neutron transparent.

Once irradiated, it is preferable to pack and dispatch doses of the radioactive microspheres to hospitals for use in medical procedures fairly quickly before the amount of radiation provided by the yttrium-90 decays below a therapeutically useful radioactivity level. However, if the window of opportunity is missed, the microspheres may be sterilised, put back into storage, and re-used later.

Advantageously, the silica microspheres manufactured in accordance with some embodiments of the present method have an apparent-density-when immersed in the range of approximately 1.2 g·cm⁻³ to 2.2 g·cm⁻³. This compares very favourably with the prior art ceramic-based microspheres sold under the Therasphere® trademark, which typically have an apparent-density-when immersed of approximately 3.0 g·cm⁻³. Generally, for medical usages, it is highly preferable for the apparent-density-when immersed to be less than 1.5 g·cm⁻³ and preferable as close to 1.1 g·cm⁻³ as possible. This is because higher densities increase the risk that the microspheres may sediment too rapidly out of the blood plasma before they have reached their intended site (e.g. the tumour if being used in SIRT). Additionally, higher densities increase the risk that the microspheres may not distribute evenly in the target organ and may accumulate excessively in non-target parts of the organ, which decreases the amount of radiation that reaches the cancer in the target organ and may cause other complications. Denser microspheres, particularly microspheres with a density greater than about 2.3 g·cm⁻³, can be difficult to deliver through infusion tubing as they exhibit a higher propensity to settle within the tubing unless the injection force is great and the flow rate of the suspending liquid is high. High pressures and fast delivery flow rates are contra-indicated when infusing radioactive microspheres into the hepatic artery of patients due to the risk of the microspheres refluxing back into blood vessels such as the gastro-duodenal artery, splenic artery, and left gastric artery, which can result in undesirable consequences. In contrast, lighter microspheres, such as those manufactured by some embodiments of the present method, distribute well within the liver.

Hence, some preferred embodiments of the present invention have the potential to offer (for the first time in the world to the best of the inventors' knowledge) a method of manufacturing microspheres that combines the major benefits of the two commercially available prior art particles. In other words, the microspheres manufactured by some embodiments of the present method have the potential to exhibit a low density that is comparable to that of SIR-Spheres® and combine this with ease of manufacturing, storage and use that is comparable to that offered by Theraspheres®.

Throughout this detailed description various theories have been espoused regarding the reasons for taking various steps and/or the underlying mechanisms that are believed to give rise to certain results or properties, along with various calculated values. Whilst these theories and calculated values are believed by the inventors as at the priority date of this application to be correct, they are nevertheless merely being postulated as possibilities and they are not to be construed as limiting the invention in any manner.

While a number of preferred embodiments have been described, it will be appreciated by persons skilled in the art that numerous variations and/or modifications may be made to the invention without departing from the spirit or scope of the invention as broadly described. The present embodiments are, therefore, to be considered in all respects as illustrative and not restrictive.

1. A method of manufacturing silica microspheres, the method including the steps of:
mixing acid and water to form a mixture;
adding a silicon alkoxide to the mixture so as to precipitate microspheres;
allowing the microspheres to settle into a sediment and removing a supernatant liquid; and
immersing the microspheres in acid.

2. The method according to claim 1 including monitoring the temperature of the mixture whilst the microspheres are precipitating and waiting until the temperature is at or near a peak before taking the steps of: allowing the mixture to settle; removing the supernatant liquid; and immersing the microspheres in acid.

3. The method according to claim 1 including allowing the microspheres to precipitate for a period of between 5 and 25 minutes and then taking the steps of: allowing the mixture to settle; removing the supernatant liquid and immersing the microspheres in acid.

4. The method according to claim 1 further including, after immersing the microspheres in acid, allowing the microspheres to settle into a sediment and removing a supernatant liquid.

5. The method according to claim 4 wherein the steps of:
immersing the microspheres in acid;
allowing the microspheres to settle; and
removing the supernatant liquid are each repeated at least once.

6. The method according to claim 4 further including immersing the microspheres in water, allowing the microspheres to settle into a sediment and removing a supernatant liquid.

7. The method according to claim 6 wherein the steps of:
immersing the microspheres in water;
allowing the microspheres to settle; and
removing the supernatant liquid
are each repeated at least once.

8. The method according to claim 4 further including immersing the microspheres in an alkali, allowing the microspheres to settle into a sediment and removing a supernatant liquid.

9. The method according to claim 8 wherein the steps of:
immersing the microspheres in an alkali;
allowing the microspheres to settle into a sediment; and
removing a supernatant liquid
are each repeated at least once.

10. The method according to claim 8 wherein the alkali is ammonia.

11. The method according to claim 8 wherein the alkali is sodium hydroxide.

12. The method according to claim 1 further including drying the microspheres at temperatures of less than 200° C.

13. The method according to claim 12 wherein the step of drying the microspheres includes:
immersing the microspheres in water in a container and placing the container in a water bath having a temperature of approximately 90° C. to 100° C. for at least 30 minutes;
removing a majority of the supernatant water so as to leave an approximately 1 mm to 5 mm layer of water above the microspheres;
placing the microspheres within the container in a dryer at a temperature of between 100° C. and 110° C. for at least 10 hours;
progressively raising the temperature of the dryer to a temperature of between 140° C. and 160° C. over a period of approximately 1 hour; and
maintaining the microspheres within the container in the dryer at a temperature of between 140° C. and 160° C. for at least 7 hours.

14. The method according to claim 1 further including drying the microspheres at temperatures of between 200° C. and 400° C.

15. The method according to claim 14 wherein the step of drying the microspheres includes:
drying the microspheres in air at an ambient temperature for between 12 and 36 hours;
placing microspheres in a dryer that is progressively heated to a temperature of between 250° C. and 350° C. over a period of 20 minutes to 40 minutes; and
maintaining the microspheres in the dryer at a temperature of between 250° C. and 350° C. for 30 to 90 minutes.

16. The method according to claim 1 further including infusing a radionuclide into the microspheres.

17. The method according to claim 16 wherein the radionuclide is a material containing yttrium.

18. The method according to claim 17 wherein the step of infusing a radionuclide into the microspheres includes:
placing the microspheres into a container;
mixing the microspheres with a yttrium-89 nitrate solution;
placing the container into a water bath having a temperature of approximately 90° C. to 100° C. for at least 30 minutes.
leaving the container in the water bath for at least 10 hours whilst allowing the water bath to cool;
removing the supernatant yttrium-89 nitrate solution;
adding water, allowing the microspheres to settle and removing supernatant water; and
calcining the microspheres.

19. The method according to claim 16 wherein the step of infusing a radionuclide into the microspheres is repeated at least once.

20. The method according to claim 19 wherein, prior to a repeating of the step of infusing a radionuclide into the microspheres, the microspheres are calcined at a temperature of between 300° C. and 500° C.

21. The method according to claim 18 wherein the step of calcining the microspheres includes:
allowing the microspheres to cool;
placing the microspheres into a dryer having a temperature of approximately 100° C. to 110° C. for at least 10 hours;
placing the microspheres into a furnace and heating the furnace at a rate of approximately 150° C. to 250° C. per hour to a target temperature of approximately 600° C. to 950° C.; and
maintaining the microspheres at the target temperature for 30 to 90 minutes.

22. The method according to claim 18 further including exposing yttrium-89 infused microspheres to neutron radiation so as to form yttrium-90 infused microspheres.

23. The method according to claim 22 wherein, prior to exposing the yttrium-89 infused microspheres to neutron radiation, the yttrium-89 infused microspheres are stored whilst in a non-radioactive state.

24. The method according to claim 1 wherein the silicon alkoxide is tetra ethyl ortho silicate (TEOS).

25. The method according to claim 1 wherein the acid is acetic acid.

26. Silica microspheres manufactured in accordance with the method of claim 1.

27. A method of treating a patient comprising:
preparing silica microspheres in accordance with the method of claim 10;
infusing the silica microspheres with a radionuclide; and
administering the radionuclide infused microsphere to the patient.

28. The method according to claim 27 wherein the radionuclide contains yttrium.

29. The method according to claim 28 wherein the microspheres have a yttrium load by weight of between approximately 0.1% and 5%.

30. The Silica microspheres according to claim 26 wherein the microspheres are neutron transparent.

31. A method of treating a patient comprising:
preparing silica microspheres in accordance with the method of claim 10;
infusing the silica microspheres with a medicament; and
administering the medicament infused silica microspheres to the patient.

32. The silica microspheres manufactured in accordance with the method of claim 12 wherein the microspheres have an apparent-density-when immersed in the range of approximately 1.2 g·cm⁻³ to 2.2 g·cm⁻³.

33. The silica microspheres manufactured in accordance with the method of claim 14 wherein the microspheres have a total open porosity in the range of approximately 5% to 40%.
